(19) United States
(12) Patent Application Publication
(10) Pub. No.: US 2022/0249526 A1
(43) Pub. Date: Aug. 11, 2022

(54) HUMAN MILK OLIGOSACCHARIDES AND COMPOSITIONS THEREOF FOR USE IN PREVENTING, MANAGING OR TREATING SYMPTOMS RELATED TO MIGRAINE

(71) Applicant: Glycom A/S, Hørsholm (DK)

(72) Inventors: Louise Kristine Vigsnæs, Copenhagen NV (DK); Bruce McConnell, La Tour de Peilz (CH)

(21) Appl. No.: 17/618,366

(22) PCT Filed: Jun. 12, 2020
(86) PCT No.: PCT/IB2020/055517

§ 371 Date: Dec. 10, 2021

(30) Foreign Application Priority Data
Jun. 12, 2019 (DK) PA 2019 00707

(51) Int. Cl. A61K 31/702; A61K 35/20; A61P 1/14

(52) U.S. Cl. CPC A61K 31/702; A61P 1/14; A61K 35/20

(57) ABSTRACT

The invention relates to a human milk oligosaccharide (HMO) for use in, a synthetic composition comprising an HMO for use in and a method for preventing, managing or treating postdrome symptoms of migraine, and/or abdominal migraine, and/or the secondary prevention of stress/anxiety induced migraine.

### FIELD OF THE INVENTION

This invention relates to a method, compounds and compositions for the prevention, management and/or treatment of selected forms of migraine and migraine associated symptoms in a human.

### BACKGROUND OF THE INVENTION

Migraine headache is a common neurologic condition characterized by painful headaches associated with nausea, vomiting, and hypersensitivity to visual, auditory, and olfactory stimuli. Migraine attacks can cause significant pain for hours to days and can be so severe that the pain is disabling. Symptoms known as aura may occur before or with the headache. These can include flashes of light, blind spots, or tingling on one side of the face or in the arm or leg.

The worldwide prevalence of migraine headache is about 10%, however, it is slightly higher in the United States (about 12%). The Global Burden of Disease Study ranked migraine as the seventh most common disabling pathology among 289 diseases. Women experience migraine headache at a greater rate (1-17%) as compared to men. Migraine headache sufferers often experience substantial decreases in work productivity and function. This results in decreased quality of life for the patient and includes high financial burdens on individuals and employers. A conservative estimate of the migraine-related costs in the United States is estimated to be 78 billion dollars per year (Thompson et al. J. Clin. Pharm. Ther. 42, 394 (2017)).

Migraine headache is a neurovascular disorder involving cortical spreading depression, neurogenic inflammation, and dysfunction in cranial vascular contractility. Certain triggers can provoke a migraine attack. These include hormonal changes, emotional triggers, physical causes (like intense physical exertion), diet factors (e.g. alcohol, caffeine, nitrite), medication, and environmental triggers, e.g. bright light, strong smell or loud sound.

Another form of migraine is abdominal migraine. This is a form of migraine which mainly occurs in children but it can occur in adults. The symptoms are mainly abdominal pain coupled with one or more of loss of appetite, nausea, vomiting and pallor. The pain associated with abdominal migraine is generally located in the middle of the abdomen. Attacks generally last between 1 to 72 hours. The abdominal migraine is an episodic syndrome which may be associated with migraine headache. Children who suffer from abdominal migraine usually grow out of it in their teens. However, they then very often develop migraine headaches.

The exact mechanism of the various forms of migraine is still not fully understood and complete preventive and attack therapy is still not available. It is believed that the fundamental mechanism of migraine headache attack involves activation of the trigeminovascular system. Through a trigger mechanism, vasodilatation of the dural and pial blood vessels occurs, which can stimulate the perivascular trigeminal primary nerve endings. The activated nociceptors release neuropeptides at the periphery, including calcitonin generated peptide, substance P and neurokinin A. These substances cause inflammation of the trigeminal nerve. When the inflammation and blood vessels interact, the blood vessels dilate, leading to pain. The brain itself does not contain neurons that are sensitive to pain. Pain arises when pressure activates the nerves sensitive to pain in the tissues covering the brain or in the muscles and blood vessels around the face, neck and scalp.

The mechanisms associated with abdominal migraine are unknown. However, it is believed that genetic influences and differences in immune and neuronal structures within the bowel mucosa may be potential underlying physiological mechanisms. Also, significant differences in gut mucosal permeability between patients with abdominal migraine and controls have been observed. It is also believed that visceral hypersensitivity to distension in response to abnormalities in neurophysiology at the level of the gut, spinal cord, or higher cortical systems may be involved. This may be due to stress stimulating the central nervous system and giving rise to an abnormal effect on the gastrointestinal system through dysregulation of neuropeptide and neurotransmitter release.

Glutamate is the principal excitatory neurotransmitter in the central nervous system and plays an important role in primary afferent neurotransmission and nociception. Numerous human and animal studies suggest that glutamate and the glutamatergic system is overactive in migraine. Glutamate is an ionic form of the nonessential amino acid glutamic acid and it excites nearly every neuron contributing to primary neural transmission and pain perception. As a neurotransmitter, glutamate is synthesized from glutamine by the mitochondrial enzyme glutaminase and is stored in synaptic vesicles. During neurotransmission, it is released from the stores to the synaptic cleft and removed by the presynaptic glutamate transporter and the transporter located on the neighbouring glial cells.

Glutamate receptors are also found in the trigeminal system. One of the main glutamate ionotropic receptors is N-methyl D-aspartate (NMDA). Activation of NMDA by glutamate causes damage to cell structures and DNA causing neuronal cell death, and glutamate excitotoxicity is related to the hyperexcitability of NMDA receptors, which plays a key role in the pathophysiology of migraine. NMDA is activated or inhibited by metabolites of the kynurenine pathway.

Tryptophan is metabolized along the kynurenine and serotonin pathways, resulting in formation of kynurenine metabolites, and neuroactive serotonin and melatonin. The two pathways are unequal in their ability to degrade tryptophan with 95% of tryptophan catabolized by the kynurenine pathway and 5% catabolized by the serotonin pathways. In the kynurenine pathways, tryptophan is transformed to N-formyl-L-kynurenine by tryptophan 2,3-dioxygenase and indoleamine 2,3-dioxygenase (IDO), which are the rate-limiting enzymes of the Kynurenine pathway and are regulated by the cytokines TNF-α and IFN-γ. N-formyl-L-kynurenine can be further metabolized to L-kynurenine (L-KYN), which is the precursor of kynurenic acid (KYNA). L-KYN can also be degraded to anthranilic acid or to 3-hydroxy-L-kynurenine. Anthranilic acid and 3-hydroxy-L-kynurenine are then further transformed to 3-hydroxyanthranilic acid, which is metabolized to quinolinic acid. Among the kynurenine pathway metabolites, many compounds are biologically active. 3-hydroxy-L-kynurenine and 3-hydroxyanthranilic acid are able to increase the formation of free radicals, yielding oxidative stress. On the contrary, KYNA has a neuroprotective function, since it acts as an antagonist for NMDA.

In the serotonin pathway, tryptophan is transformed to 5-Hydroxytryptophan by tryptophan hydroxylase. 5-Hydroxytryptophan can then be metabolized to serotonin, which can further be metabolized to melatonin by serotonin N-acetyl transferase. Melatonin has a very important role in tryptophan metabolism because it affects the two key enzymes of the two pathways; IDO and serotonin N-acetyl transferase. Melatonin has shown to induce the expression of IDO while decrease the expression of serotonin N-acetyl transferase.

Abnormalities of both the kynurenine and serotonin pathway have been reported in migraine suffers. Lower levels of serotonin and melatonin have been measured in patients with migraine compared to control, and a study has shown that chronic migraine patients had an astonishing elevation of anthranilic acid, with a decline in all other kynurenines especially KYNA. The reduction in the levels of KYNA can lead to overactivation of NMDA and low serotonin levels can lead to lower levels of melatonin, which again can cause an imbalance in the kynurenine metabolites. These events lead to migraine headaches. In addition, depletion of tryptophan has shown to increases nausea, headache and photophobia in migraine patients (Drummond Cephalalgia 26, 1225 (2006); Curto et al. J. Headache Pain 17:47 (2016)). This suggests that the level and catabolism of tryptophan is dysregulated in patients with migraine.

Since tryptophan is an essential amino acid with an estimated dietary requirement of 5 mg/kg/day and it is the limiting amino acid in nearly all protein sources, dietary sources of tryptophan may not be enough to obtain a regulated kynurenine and serotonin pathway.

The level of essential vitamins such as riboflavin, folate and pyridoxal phosphate are also important for protecting against neurotoxicity. The three B-vitamins can act through ameliorating oxidative stress, mitochondrial dysfunction, neurogenic inflammation, and glutamate excitotoxicity, and they play a key role in the tryptophan-kynurenine pathway. Hence, B vitamin insufficiency can lead to significant neurological consequences. Taking into consideration the limited vitamin absorption and utilization in 10-15% of global population, long term vitamin insufficiency could contribute to the development of multiple neurological disorders such as migraine. Although B-vitamins are present as supplements, and in a variety of foods, deficiencies still occur, mainly due to low bioavailability, and malnutrition because of insufficient food intake and unbalanced diet. Vitamin B supplements have been used in the treatment of therapy for migraine, however, in situ fortification by intestinal bacteria seems to be a better option due to the constant bioavailability of the vitamins.

Increasing evidence suggests that the intestinal microbiota play a key role in the generation of neuroinflammatory disorders. The intestinal microbiota consists of a vast bacterial community that resides primarily in the colon and lives in a symbiotic relationship with the host. The human gastrointestinal microbiota includes at least 1000 different species of bacteria, which collectively make up to 10¹⁴ bacterial cells, tenfold the number of human cells, and they encode 100-fold more unique genes than the human genome (Qin et al. Nature 464, 59 (2010)). The intestinal bacteria may directly communicate with the central nervous system by way of the vagus sensory nerve. The vagus sensory nerve is a key component of the neuro-immune and brain-gut axes through a bidirectional communication between the brain and the gastrointestinal (GI) tract.

Significant associations have also been reported between migraine and a range of inflammatory disorders such as asthma, obesity, metabolic syndrome, allergies and GI disorders such as irritable bowel syndrome and coeliac disease. These associations have been found in two directions: migraine patients have more often GI disorders compared with healthy controls, and patients with GI disorders more often suffer from migraine compared to control groups. This indicates a significant association between gut and migraine. These associations could be explained by an altered intestinal microbiota, an inflammatory immune response and an increased intestinal permeability, all of which have been found in GI disorders. An altered intestinal microbiota has been observed in migraine suffers, and people with GI disorders. This can cause an increase in the intestinal permeability. Impaired permeability allows leakage of indigestible food particles and bacterial components like liposaccharides (LPS) into the bloodstream. The present of LPS and detrimental metabolites in the blood stream can affect the blood-brain barrier. A disruption of the blood-brain barrier can lead to neuroinflammation and trigger a response provoking migraine (Lankarani et al. Middle East J. Dig. Dis. 9, 139 (2017)). In addition, gamma-aminobutyric acid (GABA) is a potent inhibitory neurotransmitter that can both be created and destroyed by intestinal bacteria. Hence, an imbalance in the metabolism of GABA could be linked to the occurrence and frequency of migraine attacks.

At present, there is no cure for migraine. Typically, pain is treated using pain-relieving medications such as aspirin, ibuprofen and triptans. Generally, these medications are either useful only for mild migraine or have significant side effects such as nausea, dizziness, drowsiness, muscle weakness, strokes and heart attacks. Preventative medication for migraine headaches is available, but none of the currently available, preventive medication stops headaches completely and they have serious side effects. Usually, these medications have been developed for other purposes such anti-depressants, anti-seizure, etc. There is no preventative medication for abdominal migraine.

Further, after the pain has subsided in a migraine attack, many patients suffer postdrome symptoms such as fatigue, body aches, trouble concentrating, dizziness and sensitivity to light. These postdrome symptoms can last for hours to days. There is no known cause of postdrome symptoms. However, it is believed that the profound changes in activity and blood flow that occur during the pain phase of the migraine attack may persist after the attack and lead to the postdrome symptoms. Recommended treatment of postdrome symptoms includes drinking plenty of water and resting or undertaking calming activities.

WO 2017/198276 describes synthetic compositions and methods for the prophylaxis or treatment of serotonin and/or tryptophan dysregulation in a human using human milk oligosaccharides, particularly for improving gut motility. Conditions mentioned are depression, anxiety, anger, being unusually sensitive to pain, carbohydrate cravings and binge eating, constipation, digestive disorders, feeling glum from lack of sunlight, feeling overly dependent on others, feeling overwhelmed, hypervigilance, insomnia, joylessness, low self-esteem, migraines, poor cognitive function and tinnitus.

There are currently no effective interventions for preventing, managing or treating postdrome symptoms of migraine and/or abdominal migraine which are effective and have limited side effects; especially for children. Therefore, there is a great need for methods and compounds for preventing, managing or treating postdrome symptoms of migraine and/or abdominal migraine in humans which are effective, safe and well tolerated.

### SUMMARY OF THE INVENTION

A first aspect of the invention relates to a human milk oligosaccharide (HMO) for use in:

preventing, managing or treating postdrome symptoms of migraine in a human,

preventing, managing or treating abdominal migraine in a human, and/or

the secondary prevention of stress and/or anxiety induced migraine in a high-risk human patient.

A second aspect of the invention relates to a synthetic composition for use in

preventing, managing or treating postdrome symptoms of migraine in a human,

preventing, managing or treating abdominal migraine in a human, and/or

the secondary prevention of stress and/or anxiety induced migraine in a high-risk human patient, the composition comprising at least one human milk oligosaccharide (HMO).

Preferably the synthetic composition contains an amount of 1 g to 15 g of the HMO; more preferably 2 g to 10 g. For example, the synthetic composition may contain 3 g to 7 g of the HMO.

The synthetic composition may contain a bifidobacteria; for example, Bifidobacterium longum and/or Bifidobacterium bifidum. The synthetic composition may also comprise a source of magnesium, a vitamin B source, melatonin, coenzyme Q10, and/or an omega-3 long-chain fatty acid. For example, the synthetic composition may comprise a vitamin B source and/or an omega-3 long-chain fatty acid.

A third aspect of the invention relates to a method for preventing, managing or treating postdrome symptoms of migraine in a human, the method comprising administering to the human an effective amount of at least one human milk oligosaccharide (HMO).

Preferably the postdrome symptoms are one or more of fatigue, body aches, trouble concentrating, dizziness and sensitivity to light. More preferably, fatigue is reduced.

A fourth aspect of the invention relates to a method for preventing, managing or treating abdominal migraine in a human, the method comprising administering to the human an effective amount of at least one human milk oligosaccharide (HMO).

The human at risk of our suffering from abdominal migraine is preferably a child. The human can suffer from an impaired gastrointestinal barrier and the amount of HMO administered is effective to increase gastrointestinal barrier function.

The human may suffer from chronic migraine or have another disorder, e.g. an inflammatory disorder (such as asthma), obesity, metabolic syndrome, allergies and/or a disease or condition involving gastrointestinal symptoms. For example, the disease or condition involving gastrointestinal symptoms can be an autoimmune disease such as coeliac disease, irritable bowel syndrome, an allergy and/or a food intolerance such as non-coeliac gluten/wheat intolerance. The human may also suffer from abnormal serotonin or tryptophan metabolism.

A fifth aspect of the invention relates to a method for the secondary prevention of stress and/or anxiety induced migraine in a high-risk human patient, the method comprising prophylactically administering to the human an effective amount of at least one human milk oligosaccharide (HMO).

Preferably, the human is administered the HMO for a period of at least 1 week, more preferably for at least 2 weeks.

Preferably, the human is administered an amount of 1 g to 15 g per day of the HMO; more preferably 2 g to 10 g per day. For example, the human may be administered 3 g to 7 g per day. The human may be administered higher doses during an initial phase and lower doses during a second, maintenance phase.

A sixth aspect of the invention is a pack for use in in:

preventing, managing or treating postdrome symptoms of migraine in a human,

preventing, managing or treating abdominal migraine in a human, and/or

the secondary prevention of stress and/or anxiety induced migraine in a high-risk patient,

the pack comprising at least 14 individual daily doses of an effective amount of at least one human milk oligosaccharide (HMO).

Preferably each dose in the pack contains about 1 g to 15 g of the human milk oligosaccharide, preferably 2 g to 10 g, more preferably 3 g to 7 g. Further the pack preferably comprises at least about 21 daily doses; for example, about 28 daily doses.

The HMO can be a neutral HMO or an acidic HMO. The neutral HMO can be one or more fucosylated HMOs or one or more non-fucosylated HMOs. Preferably, the HMO is selected from 2′-FL, 3-FL, DFL, LNT, LNnT, 3′-SL, 6′-SL, LNFP-I or a mixture thereof. Preferably, the HMO comprises 2′-FL and at least one of LNnT and LNT; at least one of 2′-FL and DFL and at least one of LNnT and LNT (e.g. 2′-FL, DFL and at least one of LNnT and LNT); 2′-FL and 6′-SL; DFL and 6′-SL; 2′-FL, DFL and 6′-SL; 2′-FL, 6′-SL and at least one of LNnT and LNT; and 2′-FL, DFL, 6′-SL and at least one of LNnT and LNT.

The human may be further administered a source of magnesium, a vitamin B source, melatonin, coenzyme Q10, and/or an omega-3 long-chain fatty acid and/or a probiotic bacterium, e.g. one or more bifidobacteria) species. Preferably the human is further administered a vitamin B source and/or an omega-3 long-chain fatty acid.

### DETAILED DESCRIPTION OF THE INVENTION

It has now been surprisingly found that oral or enteral administration of one or more HMOs to humans suffering from postdrome symptoms of migraine and/or abdominal migraine, or at high risk of stress/anxiety induced migraine, reduces or prevents occurrence and/or symptom severity. HMOs have an excellent safety and tolerance profile and therefore they are ideally suited for use in the prevention, management and treatment of postdrome symptoms of migraine and/or abdominal migraine. In addition, administration of HMOs to humans preferentially creates a beneficial intestinal microbiota by increasing the abundance Bifidobacterium of the B. adolescentis phylogenetic group, Bifidobacterium longum and/or Bifidobacterium bifidum. These bacteria produce lactate and acetate which in turn can be converted into butyrate by butyrate-producing bacteria. As an outcome, the gastrointestinal permeability and inflammation is diminished. Also, the bifidobacteria are able to synthesise B-vitamins such as riboflavin and folate de novo, ensuring its constant bioavailability, and can secrete neuromodulators such as GABA. In addition, species of bifidobacteria are able to synthesize tryptophan, and can impact immune regulation and expression of different immune markers such as IFN-γ and TNF-α. The kynurenine pathway is regulated by IFN-γ and TNF-α, hence selective stimulation of bifidobacteria can affect tryptophan metabolism and help regulate the serotonin and kynurenine pathways. Furthermore, fucosylated HMOs can stimulate the central nervous system through the afferent vagus nerve (Vazquez et al. PLoS ONE 11: e0166070 (2016)).

Human milk oligosaccharides (HMOs) are a heterogeneous mixture of soluble glycans found in human milk. They are the third most abundant solid component after lactose and lipids in human milk and are present in concentrations of 5-25 g/l ((Bode: Human milk oligosaccharides and their beneficial effects, in: Handbook of dietary and nutritional aspects of human breast milk (Zibadi et al., eds.), pp. 515-31, Wageningen Academic Publishers (2013)). HMOs are resistant to enzymatic hydrolysis in the small intestine and are thus largely undigested and unabsorbed and reach the colon intact. The majority of HMOs that reach the colon serve as substrates to shape the gut ecosystem by selectively stimulating the growth of specific bacteria. HMOs are believed to substantially modulate the infant gut microbiota and play a decisive role in the differences in the microbiota of formula-fed and breast-fed infants. HMOs are also able to substantially modulate to intestinal bacteria of older children and adults and to have a positive effect on gut health.

In this specification, the following terms have the following meanings that are applicable to all embodiments described herein, unless specified otherwise:

“Abdominal migraine” means a condition marked by episodic moderate to severe abdominal pain. The pain usually lasts from 1 hour to three days. There is typically complete normality between episodes. Abdominal migraine may have an early symptom (prodrome) indicating the onset of a disease or illness, e.g. constipation, mood changes food cravings, etc. The pain typically begins in the middle of the abdomen (belly) and is usually accompanied by symptoms such as little desire to eat, nausea, and vomiting. Other symptoms can include sensitivity to light, sensitivity to sound and dizziness. Abdominal migraine may be often followed by one or more symptoms that occurs after the attack (postdrome) and last from a few hours to about 2-3 days, e.g. fatigue, mental confusion, skin and scalp sensitivity, mood change, etc. Abdominal migraine occurs mainly in children. In particular, the invention relates to migraine patients that are categorised as having abdominal migraine according to the criteria defined in The International Classification of Headache Disorders, 3rd edition (https://ichd-3.org/1-migraine/1-6-episodic-syndromes-that-may-be-associated-with-migraine/1-6-1-recurrent-gastrointestinal-disturbance/1-6-1-2-abdominal-migraine/). “Bifidobacterium of the B. adolescentis phylogenetic group” means a bacterium selected from a group consisting of Bifidobacterium adolescentis, Bifidobacterium angulatum, Bifidobacterium catenulatum, Bifidobacterium pseudocatenulatum, Bifidobacterium kashiwanohense, Bifidobacterium dentum and Bifidobacterium stercoris (Duranti et al. Appl. Environ. Microbiol. 79, 336 (2013), Bottacini et al. Microbial Cell Fact. 13:54 (2014)). Preferably, a Bifidobacterium of the B. adolescentis phylogenetic group is Bifidobacterium adolescentis and/or Bifidobacterium pseudocatenulatum.

“Chronic migraine” means a migraine condition in which patients have at least 15 days with headache per month for at least 3 months. Chronic migraine is a more extreme version of recurrent migraine.

“Effective amount” means an amount of an HMO sufficient to render a desired outcome in a human. An effective amount can be administered in one or more doses to achieve the desired outcome.

“Enteral administration” means any conventional form for delivery of a composition to a human that causes the deposition of the composition in the gastrointestinal tract (including the stomach). Methods of enteral administration include feeding through a naso-gastric tube or jejunum tube, oral, sublingual and rectal.

“High-risk individual” means an individual who is not known to suffer from the condition, e.g. migraine, but who is genetically predisposed or who has another physiological condition, e.g. a disease or metabolic disorder, such as an inflammatory disorder e.g. asthma, obesity, metabolic syndrome; allergies; GI disorders such as irritable bowel syndrome or coeliac disease; abnormal serotonin or tryptophan metabolism, that can evoke the onset of the condition, e.g. a migraine attack.

“Human milk oligosaccharide” or “HMO” means a complex carbohydrate found in human breast milk (Urashima et al.: Milk Oligosaccharides. Nova Science Publisher (2011); Chen Adv. Carbohydr. Chem. Biochem. 72, 113 (2015)). The HMOs have a core structure comprising a lactose unit at the reducing end that can be elongated by one or more β-N-acetyl-lactosaminyl and/or one or β-more lacto-N-biosyl units, and which core structure can be substituted by an a L-fucopyranosyl and/or an a-N-acetyl-neuraminyl (sialyl) moiety. In this regard, the non-acidic (or neutral) HMOs are devoid of a sialyl residue, and the acidic HMOs have at least one sialyl residue in their structure. The non-acidic (or neutral) HMOs can be fucosylated or non-fucosylated. Examples of such neutral non-fucosylated HMOs include lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH). Examples of neutral fucosylated HMOs include 2′-fucosyllactose (2′-FL), lacto-N-fucopentaose I (LNFP-I), lacto-N-difucohexaose I (LNDFH-I), 3-fucosyllactose (3-FL), difucosyllactose (DFL), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-I), fucosyl-para-lacto-N-neohexaose II (F-pLNnH II) and fucosyl-lacto-N-neohexaose (FLNnH). Examples of acidic HMOs include 3′-sialyllactose (3′-SL), 6′-sialyllactose (6′-S L), 3-fucosyl-3′-sialyllactose (FS L), LST a, fucosyl-LST a (FLST a), LST b, fucosyl-LST b (FLST b), LST c, fucosyl-LST c (FLST c), sialyl-LNH (SLNH), sialyl-lacto-N-hexaose (SLNH), sialyl-lacto-N-neohexaose I (SLNH-I), sialyl-lacto-N-neohexaose II (SLNH-II) and disialyl-lacto-N-tetraose (DSLNT).

“Managing” a medical condition in a person means addressing specific nutritional needs of the person using diet or nutritional interventions. “Manage” and “management” have grammatically corresponding meanings.

“Microbiota”, “microflora” and “microbiome” mean a community of living microorganisms that typically inhabits a bodily organ or part, particularly the gastro-intestinal organs of humans. The most dominant members of the gastrointestinal microbiota include microorganisms of the phyla of Firmicutes, Bacteroidetes, Actinobacteria, Proteobacteria, Synergistetes, Verrucomicrobia, Fusobacteria, and Euryarchaeota; at genus level Bacteroides, Faecalibacterium, Bifidobacterium, Roseburia, Alistipes, Collinsella, Blautia, Coprococcus, Ruminococcus, Eubacterium and Dorea; at species level Bacteroides uniformis, Alistipes putredinis, Parabacteroides merdae, Ruminococcus bromii, Dorea longicatena, Bacteroides caccae, Bacteroides thetaiotaomicron, Eubacterium hallii, Ruminococcus torques, Faecalibacterium prausnitzii, Ruminococcus lactaris, Collinsella aerofaciens, Dorea formicigenerans, Bacteroides vulgatus and Roseburia intestinalis. The gastrointestinal microbiota includes the mucosa-associated microbiota, which is located in or attached to the mucus layer covering the epithelium of the gastrointestinal tract, and luminal-associated microbiota, which is found in the lumen of the gastrointestinal tract.

“Migraine” means a common disabling primary headache disorder. Migraine has two major types: 1. migraine without aura is a clinical syndrome characterized by headache with specific features and associated symptoms; 2. migraine with aura is primarily characterized by the transient focal neurological symptoms that usually precede or sometimes accompany the headache. Some patients also experience a prodromal phase, occurring hours or days before the headache and/or a postdromal phase following headache resolution. Prodromal and postdromal symptoms include hyperactivity, hypoactivity, depression, cravings for particular foods, repetitive yawning, fatigue and neck stiffness and/or pain. In general, the invention relates to subject who are diagnosed with migraine according to classification of The International Classification of Headache Disorders, 3rd edition (https://ichd-3.org/1-migraine/).

In particular, in the present context, “migraine headache” means a condition marked by episodic moderate to severe headache with throbbing pain. The pain usually lasts from two hours to three days. It may have an early symptom (prodrome) indicating the onset of a disease or illness, e.g. constipation, mood changes food cravings, etc. The migraine headache attack typically begins on one side of the head but may spread to both sides and is often accompanied by nausea, vomiting, and sensitivity to light or sound, and is sometimes preceded by an aura. Migraine headache is often followed by one or more symptoms that occurs after the attack (postdrome) and last from a few hours to about 2-3 days, e.g. fatigue, mental confusion, skin and scalp sensitivity, mood change, etc.

“Migraine patient” means an individual that suffers from abdominal migraine or migraine headache, either or not accompanied with prodrome and/or postdrome symptoms.

“Migraine postdrome” is a phase of the migraine attack which typically occurs after the end of the headache or abdominal pain phase. Not every person with migraine suffers from postdrome, but it does occur in most (approximately 80%). Symptoms of postdrome include fatigue, body aches, trouble concentrating, depression, dizziness and sensitivity to light.

“Modulating of microbiota” means exerting a modifying or controlling influence on microbiota, for example an influence leading to an increase in the indigenous intestinal abundance of Bifidobacterium, Barnesiella and/or Faecalibacterium and/or other butyrate producing bacteria. In another example, the influence may lead to a reduction of the intestinal abundance of Ruminococcus gnavus and/or Proteobacteria. “Proteobacteria” are a phylum of Gram-negative bacteria and include a wide variety of pathogenic bacteria, such as Escherichia, Salmonella, Vibrio, Helicobacter, Yersinia and many other notable genera.

“Oral administration” means any conventional form for the delivery of a composition to a human through the mouth. Accordingly, oral administration is a form of enteral administration.

“Patient” in general is an individual suffering from a disease or a pathological condition who is currently or have been in the past put under observation or control by a qualified medical professional.

“Preventing migraine” means reducing the risk of occurrence or recurrence of one or more symptoms associated with migraine, preferable two or more symptoms, more preferably all symptoms of migraine in an individual. In one embodiment, the invention relates to preventing development a chronic migraine characterised with recurrent headache in a child having abdominal migraine.

“Preventive treatment” or “prevention” means treatment given or action taken to diminish the risk of onset or recurrence of a disease, or severity of the symptoms.

“Primary prevention” means prevention of onset of a condition in an individual who is not known to suffer from the condition.

“Recurrent migraine” means that symptoms of migraine are experienced by the individual periodically, such one from once a week, once in two weeks, once a month or more seldom, with a migraine condition that lasts from around 1 hour to around 72 hours.

“Reducing migraine” means eliminating or diminishing at least one symptom associated with the migraine headache in an individual, preferably two or more symptoms, preferably all symptoms of migraine in the individual.

“Secondary prevention” means prevention of onset of a condition in a high-risk individual, or prevention of reoccurrence of symptoms in a patient who has already has the condition.

“Symptoms of migraine headache” means a headache characterised by at least one of the following:
pain on one side or both sides of the head;
pain that feels throbbing or pulsing;
sensitivity to light, sounds, and sometimes smells and touch;
nausea and vomiting;
blurred vision;
light-headedness, sometimes followed by fainting.

“Synthetic composition” means a composition which is artificially prepared and preferably means a composition containing at least one compound that is produced ex vivo chemically and/or biologically, e.g. by means of chemical reaction, enzymatic reaction or recombinantly. The synthetic composition typically comprises one or more compounds, including one or more HMOs, that are capable of preventing, managing or treating postdrome symptoms of migraine, and/or abdominal migraine in a human. Also, in some embodiments, the synthetic compositions may comprise one or more nutritionally or pharmaceutically active components which do not affect adversely the efficacy of the above-mentioned compounds. Some non-limiting embodiments of a synthetic composition of the invention are also described below.

“Therapy” means treatment given or action taken to reduce or eliminate symptoms of a disease or pathological condition.

“Treat” means to address a medical condition or disease with the objective of improving or stabilising an outcome in the person being treated or addressing an underlying nutritional need. Treat therefore includes the dietary or nutritional management of the medical condition or disease by addressing nutritional needs of the person. “Treating” and “treatment” have grammatically corresponding meanings.

In accordance with this invention, the incidence and intensity of postdrome symptoms of migraine, and/or the incidence and intensity of abdominal migraine, in a human may be reduced or prevented by administering one or more HMOs to the human. Further, the incidence and/or intensity of stress and/or anxiety induced migraine in a high-risk patient may be reduced by administering one or more HMOs to the high-risk human. The HMOs may be administered as individual compounds or in the form of a synthetic composition.

The HMOs can be isolated or enriched by well-known processes from milk(s) secreted by mammals including, but not limited to human, bovine, ovine, porcine, or caprine species. The HMOs can also be produced by well-known processes using microbial fermentation, enzymatic processes, chemical synthesis, or combinations of these technologies. As examples, using chemistry LNnT can be made as described in WO 2011/100980 and WO 2013/044928, LNT can be synthesized as described in WO 2012/155916 and WO 2013/044928, a mixture of LNT and LNnT can be made as described in WO 2013/091660, 2′-FL can be made as described in WO 2010/115934 and WO 2010/115935, 3-FL can be made as described in WO 2013/139344, 6′-SL and salts thereof can be made as described in WO 2010/100979, sialylated oligosaccharides can be made as described in WO 2012/113404 and mixtures of human milk oligosaccharides can be made as described in WO 2012/113405. As examples of enzymatic production, sialylated oligosaccharides can be made as described in WO 2012/007588, fucosylated oligosaccharides can be made as described in WO 2012/127410, and advantageously diversified blends of human milk oligosaccharides can be made as described in WO 2012/156897 and WO 2012/156898. Descriptions of biotechnological methods to make core human milk oligosaccharides, optionally substituted by fucose or sialic acid, using genetically modified E. coli. can be found in WO 01/04341 and WO 2007/101862.

The HMO in any of the above aspects may be a single HMO or a mixture of any HMOs suitable for the purpose of the invention.

In one embodiment, the mixture comprises, consists of or consists essentially of, neutral HMOs, preferably at least a first neutral HMO and at least a second neutral HMO, wherein the first neutral HMO is a fucosylated neutral HMO and the second neutral HMO is a core HMO (also referred to as non-fucosylated neutral HMO). Particularly, the mixture of HMOs may contain a fucosylated HMO selected from the list consisting of 2′-FL, 3-FL, DFL, LNFP-I, LNFP-II, LNFP-III, LNFP-V, LNDFH-I, LNDFH-II, LNDFH-III, FLNH-I, FLNH-II, FLNnH, FpLNH-I and F-pLNnH II, and a core HMO selected from the list consisting of LNT, LNnT, LNH, LNnH, pLNH and pLNnH. More preferably, the mixture of neutral HMOs contains, consists of, or consists essentially of, a fucosylated HMO selected from the list consisting of 2′-FL, 3-FL and DFL, and a core HMO selected from the list consisting of LNT and LNnT; advantageously the mixture comprises, consists of or consists essentially of, 2′-FL and at least one of LNnT and LNT; or at least one of 2′-FL and DFL and at least one of LNnT and LNT; or 2′-FL, DFL and at least one of LNnT and LNT.

In other embodiment, the mixture comprises, consists of or consists essentially of, at least a first (acidic) HMO and at least a second (neutral) HMO, wherein the first (acidic) HMO is selected from the list consisting of 3′-SL, 6′-SL and FSL and the second (neutral) HMO is selected from the list consisting of 2′-FL, 3-FL, DFL, LNT and LNnT; advantageously the mixture comprises, consists of or essentially consists of, 2′-FL and 6′-SL; or 6′-SL and at least one of 2′-FL and DFL; or 2′-FL, 6′-SL and at least one of LNnT and LNT; or 2′-FL, DFL, 6′-SL and at least one of LNnT and/or LNT.

In one embodiment, the synthetic composition can be in the form of a nutritional composition. For example, the nutritional composition can be a food composition, a rehydration solution, a medical food or food for special medical purposes, a nutritional supplement and the like. The nutritional composition can contain sources of protein, lipids and/or digestible carbohydrates and can be in powdered or liquid forms. The composition can be designed to be the sole source of nutrition or as a nutritional supplement.

Suitable protein sources include milk proteins, soy protein, rice protein, pea protein and oat protein, or mixtures thereof. Milk proteins can be in the form of milk protein concentrates, milk protein isolates, whey protein or casein, or mixtures of both. The protein can be whole protein or hydrolysed protein, either partially hydrolysed or extensively hydrolysed. Hydrolysed protein offers the advantage of easier digestion which can be important for humans with inflamed or compromised GI tracts. The protein can also be provided in the form of free amino acids. The protein can comprise about 5% to about 30% of the energy of the nutritional composition, normally about 10% to 20%.

The protein source can be a source of glutamine, threonine, cysteine, serine, proline, or a combination of these amino acids. The glutamine source can be a glutamine dipeptide and/or a glutamine enriched protein. Glutamine can be included due to the use of glutamine by enterocytes as an energy source. Threonine, serine and proline are important amino acids for the production of mucin. Mucin coats the GI tract and can improve intestinal barrier function and mucosal healing. Cysteine is a major precursor of glutathione, which is key for the antioxidant defences of the body.

Suitable digestible carbohydrates include maltodextrin, hydrolysed or modified starch or corn starch, glucose polymers, corn syrup, corn syrup solids, high fructose corn syrup, rice-derived carbohydrates, pea-derived carbohydrates, potato-derived carbohydrates, tapioca, sucrose, glucose, fructose, sucrose, lactose, honey, sugar alcohols (e.g., maltitol, erythritol, sorbitol), or mixtures thereof. Preferably the composition is free from added lactose. Generally digestible carbohydrates provide about 35% to about 55% of the energy of the nutritional composition. A particularly suitable digestible carbohydrate is a low dextrose equivalent (DE) maltodextrin.

Suitable lipids include medium chain triglycerides (MCT) and long chain triglycerides (LCT). Preferably the lipid is a mixture of MCTs and LCTs. For example, MCTs can comprise about 30% to about 70% by weight of the lipids, more specifically about 50% to about 60% by weight. MCTs offer the advantage of easier digestion which can be important for humans with inflamed or compromised GI tracts. Generally, the lipids provide about 35% to about 50% of the energy of the nutritional composition. The lipids can contain essential fatty acids (omega-3 and omega-6 fatty acids). Preferably these polyunsaturated fatty acids provide less than about 30% of total energy of the lipid source.

Suitable sources of long chain triglycerides are rapeseed oil, sunflower seed oil, palm oil, soy oil, milk fat, corn oil, high oleic oils, and soy lecithin. Fractionated coconut oils are a suitable source of medium chain triglycerides. The lipid profile of the nutritional composition is preferably designed to have a polyunsaturated fatty acid omega-3 (n-3) to omega-3 (n-6) ratio of about 4:1 to about 10:1. For example, the n-3 to n-6 fatty acid ratio can be about 6:1 to about 9:1. The polyunsaturated fatty acid may consist of an omega-3 fatty acid.

The nutritional composition may also include vitamins and minerals. If the nutritional composition is intended to be a sole source of nutrition, it preferably includes a complete vitamin and mineral profile. Examples of vitamins include vitamins A, B-complex (such as B1, B2, B6 and B12), C, D, E and K, niacin and acid vitamins such as pantothenic acid, folate or folic acid, and biotin. Examples of minerals include calcium, iron, zinc, magnesium, iodine, copper, phosphorus, manganese, potassium, chromium, molybdenum, selenium, nickel, tin, silicon, vanadium and boron. A source of magnesium, for example magnesium dicitrate (600 mg), may reduce migraine occurrence and intensity. The nutritional composition may also include coenzyme Q10.

The nutritional composition can also include a carotenoid such as lutein, lycopene, zeaxanthin, and beta-carotene. The total amount of carotenoid included can vary from about 0.001 μg/ml to about 10 μg/ml. Lutein can be included in an amount of from about 0.001 μg/ml to about 10 μg/ml, preferably from about 0.044 μg/ml to about 5 μg/ml of lutein. Lycopene can be included in an amount from about 0.001 μg/ml to about 10 μg/ml, preferably about 0.0185 μg/ml to about 5 μg/ml of lycopene. Beta-carotene can comprise from about 0.001 μg/ml to about 10 mg/ml, for example about 0.034 μg/ml to about 5 μg/ml of beta-carotene.

The nutritional composition preferably also contains reduced concentrations of sodium; for example, from about 300 mg/I to about 400 mg/I. The remaining electrolytes can be present in concentrations set to meet needs without providing an undue renal solute burden on kidney function. For example, potassium is preferably present in a range of about 1180 to about 1300 mg/I; and chloride is preferably present in a range of about 680 to about 800 mg/I.

The nutritional composition can also contain various other conventional ingredients such as preservatives, emulsifying agents, thickening agents, buffers, fibres and prebiotics (e.g. fructooligosaccharides, galactooligosaccharides), probiotics (e.g. B. animalis subsp. lactis BB-12, B. lactis HN019, B. lactis Bi07, B. lactis W52, B. infantis ATCC 15697, B. bifidum, L. rhamnosus GG, L. rhamnosus HNOOI, L. acidophilus LA-5, L. acidophilus NCFM, L. brevis W63, L. casei W56, L. salivarius W24, L. fermentum CECT5716, B. longum BB536, B. longum AH1205, B. longum AH1206, B. breve M-16V, L. reuteri ATCC 55730, L. reuteri ATCC PTA-6485, L. reuteri DSM 17938), antioxidant/anti-inflammatory compounds including tocopherols, carotenoids, ascorbate/vitamin C, ascorbyl palmitate, polyphenols, glutathione, and superoxide dismutase (melon), other bioactive factors (e.g. growth hormones, cytokines, TFG-β), colorants, flavours, and stabilisers, lubricants, and so forth.

The nutritional composition can be formulated as a soluble powder, a liquid concentrate, or a ready-to-use formulation. The composition can be fed to a human in need via a nasogastric tube or orally. Various flavours, fibres and other additives can also be present.

The nutritional compositions can be prepared by any commonly used manufacturing techniques for preparing nutritional compositions in solid or liquid form. For example, the composition can be prepared by combining various feed solutions. A protein-in-fat feed solution can be prepared by heating and mixing the lipid source and then adding an emulsifier (e.g. lecithin), fat soluble vitamins, and at least a portion of the protein source while heating and stirring. A carbohydrate feed solution is then prepared by adding minerals, trace and ultra-trace minerals, thickening or suspending agents to water while heating and stirring. The resulting solution is held for 10 minutes with continued heat and agitation before adding carbohydrates (e.g. the HMOs and digestible carbohydrate sources). The resulting feed solutions are then blended together while heating and agitating and the pH adjusted to 6.6-7.0, after which the composition is subjected to high-temperature short-time processing during which the composition is heat treated, emulsified and homogenized, and then allowed to cool. Water soluble vitamins and ascorbic acid are added, the pH is adjusted to the desired range if necessary, flavours are added, and water is added to achieve the desired total solid level.

For a liquid product, the resulting solution can then be aseptically packed to form an aseptically packaged nutritional composition. In this form, the nutritional composition can be in ready-to-feed or concentrated liquid form. Alternatively, the composition can be spray-dried and processed and packaged as a reconstitutable powder.

When the nutritional product is a ready-to-feed nutritional liquid, it may be preferred that the total concentration of HMOs in the liquid, by weight of the liquid, is from about 0.1% to about 1.5%, including from about 0.21% to about 1.0%, for example from about 0.3% to about 0.7%. When the nutritional product is a concentrated nutritional liquid, it may be preferred that the total concentration of HMOs in the liquid, by weight of the liquid, is from about 0.2% to about 3.0%, including from about 0.4% to about 2.0%, For example from about 0.6% to about 1.5%.

In another embodiment, the nutritional composition is in a unit dosage form. The unit dosage form can contain an acceptable food-grade carrier, e.g. phosphate buffered saline solution, mixtures of ethanol in water, water and emulsions such as an oil/water or water/oil emulsion, as well as various wetting agents or excipients. The unit dosage form can also contain other materials that do not produce an adverse, allergic or otherwise unwanted reaction when administered to a human. The carriers and other materials can include solvents, dispersants, coatings, absorption promoting agents, controlled release agents, and one or more inert excipients, such as starches, polyols, granulating agents, microcrystalline cellulose, diluents, lubricants, binders, and disintegrating agents.

A unit dosage form can be administered orally, e.g. as a tablet, capsule, or pellet containing a predetermined amount of the mixture, or as a powder or granules containing a predetermined concentration of the mixture or a gel, paste, solution, suspension, emulsion, syrup, bolus, electuary, or slurry, in an aqueous or non-aqueous liquid, containing a predetermined concentration of the mixture. An orally administered composition can include one or more binders, lubricants, inert diluents, flavouring agents, and humectants. An orally administered composition such as a tablet can optionally be coated and can be formulated to provide sustained, delayed or controlled release of the HMO.

The unit dosage form can also be administered by naso-gastric tube or direct infusion into the GI tract or stomach.

The unit dosage form can also include therapeutic agents such as antibiotics, probiotics, a source of magnesium, a source of B vitamins, melatonin, coenzyme Q10, omega-3 polyunsaturated fatty acids, analgesics, and anti-inflammatory agents. The proper dosage of such a composition for a human can be determined in a conventional manner, based upon factors such as the human's condition, immune status, body weight and age. In some cases, the dosage will be at a concentration similar to that found for the HMOs of the composition in human breast milk. The required amount would generally be in the range from about 1 g to about 15 g per day, in certain embodiments from about 2 g to about 10 g per day, for example about 3 g to about 7 g per day. Appropriate dose regimes can be determined by methods known to those skilled in the art.

In further embodiment, the HMO can be comprised in a pharmaceutical composition. The pharmaceutical composition can contain a pharmaceutically acceptable carrier, e.g. phosphate buffered saline solution, mixtures of ethanol in water, water and emulsions such as an oil/water or water/oil emulsion, as well as various wetting agents or excipients. The pharmaceutical composition can also contain other materials that do not produce an adverse, allergic or otherwise unwanted reaction when administered to patients. The carriers and other materials can include solvents, dispersants, coatings, absorption promoting agents, controlled release agents, and one or more inert excipients, such as starches, polyols, granulating agents, microcrystalline cellulose, diluents, lubricants, binders, and disintegrating agents.

The pharmaceutical compositions can be administered orally, e.g. as a tablet, capsule, or pellet containing a predetermined amount, or as a powder or granules containing a predetermined concentration or a gel, paste, solution, suspension, emulsion, syrup, bolus, electuary, or slurry, in an aqueous or non-aqueous liquid, containing a predetermined concentration. Orally administered compositions can include binders, lubricants, inert diluents, flavouring agents, and humectants. Orally administered compositions such as tablets can optionally be coated and can be formulated to provide sustained, delayed or controlled release of the mixture therein.

The pharmaceutical compositions can also be administered by rectal suppository, aerosol tube, naso-gastric tube or direct infusion into the GI tract or stomach.

The pharmaceutical compositions can also include therapeutic agents such as antibiotics, probiotics, analgesics, a source of magnesium, a source of B vitamins, melatonin, coenzyme Q10, omega-3 polyunsaturated fatty acids and anti-inflammatory agents. The proper dosage of these compositions for a human can be determined in a conventional manner, based upon factors such condition, immune status, body weight and age. In some cases, the dosage of the HMOs will be at a concentration similar to that found for the HMOs in human breast milk. The required amount would generally be in the range from about 1 g to about 15 g per day, in certain embodiments from about 2 g to about 10 g per day, for example from about 3 g to about 7 g per day. Appropriate dose regimes can be determined by conventional methods.

For preventing, managing or treating postdrome symptoms of migraine, and/or the abdominal migraine in a human, and/or for the secondary prevention of stress and/or anxiety induced migraine in a high-risk patient, the amount of HMO(s) required to be administered will vary depending upon factors such as the risk and severity of the recurrent migraine, any underlying medical condition or disease, age, the form of the composition, and other medications being administered. However, the required amount can be readily set by a medical practitioner and would generally be in the range from about 1 g to about 15 g per day, in certain embodiments from about 2 g to about 10 g per day, for example from about 3 g to about 7 g per day. An appropriate dose can be determined based on several factors, including, for example, body weight and/or condition, the severity of the postdrome and/or abdominal migraine being treated, managed or prevented, other ailments and/or diseases, the incidence and/or severity of side effects and the manner of administration. Appropriate dose ranges may be determined by methods known to those skilled in the art. During an initial phase, the dosing can be higher (for example 3 g to 15 g per day, preferably 3 mg to 10 g per day). During a later maintenance phase, the dosing can be reduced (for example, 1 g to 10 g per day, preferably 2 g to 7.5 g per day).

### EXAMPLES

### Example 1

Patients of age between 18-70 years who have a history of recurrent migraine (according to the criteria of the International Classification of Headache Disorders, 3rd edition [beta version]) for at least 12 months are recruited. Patients fulfil the criteria for chronic migraine during the 28-day screening period (headache of any duration or severity on ≥15 days and headache meeting ICHD-3 beta criteria for migraine on days). Key exclusion criteria are the use of preventative medication during the 4 months before screening; the use of preventative devices such as transcranial magnetic stimulation during the 2 months before screening; and the use of opioid or barbiturate medications on more than 4 days during the screening period. Patients are also excluded if pregnant or breast-feeding women.

At an initial visit (screening), each patient is given both written and oral information about the study and the patient is asked to sign an informed consent form. Each patient is evaluated by a full review of clinical history including headache/migraine history and undergoes a physical examination and a 12-lead electrocardiogram. Blood and urine samples are collected. Equipment for faecal sampling is distributed to each patient. Patients are instructed to keep their samples in the freezer until the next visit. Patients are provided with access to an electronic diary to record headache/migraine occurrence and intensity. A 4-point Likert scale is used for pain intensity where a score of “0” implies “no pain” and a score of “3” implies “severe pain”. Patients also record associated migraine symptoms (e.g. nausea, photophobia and phonophobia), any medications used.

At a second visit (beginning of intervention) within 28 days of the first visit, eligibility criteria are checked, and eligible subjects are randomised to one of two arms. A total of 60 patients are included. Each arm has 60 patients, with one arm consuming the treatment product and one group the placebo product. The treatment product contains 5 grams of a combination of 2′-FL and LNnT while the placebo product contains 5 grams glucose. Both products are in powder form in a unit dosage container. The diary is reviewed, and an assessment is made of symptoms of physical and mental health, gastrointestinal symptoms, somatic symptoms, quality of life, anxiety and depression and faecal consistency (as measured by SF36, GSRS, PHQ12, BSFS, HADS and QoL questionnaires). Trial supplementation is distributed along with instructions on use rescue medication. The faecal samples are collected and equipment for collecting new samples is distributed. Patients are instructed to maintain their current diet.

Blood samples and urine are collected for biomarker and biobanking. The serum from the blood samples is transferred to cryotubes and stored at −80° C. The following biomarkers are measured IFN-γ, TNF-α, IL-1β, IL-8, IL-6, IL-12, IL-10, MIP-1β, hs-CRP, lipopolysaccharide binding protein, fatty acid binding protein 2, tryptase, antiflagellin, zonulin, histamine, prostaglandin 2, and cortisol. To analysis the level of metabolites of the kynurenine and serotonin pathways following compounds were measured in serum; tryptophan, L-kynurenine, kynureninic acid and serotonin. Flow cytometry are performed on blood to determine the level of immune cells.

Urine samples are stored at −80° C. Bacterial metabolites such as SOFA are analysed in urine samples using NMR.

The faecal samples are stored at −80° C. until analysis. Microbiological analysis is performed on the faecal samples using the 16S rRNA gene sequence.

At a third visit after 4 weeks, the faecal samples are collected, blood and urine samples are collected, and an assessment is made of headache/migraine occurrence and intensity, and of symptoms of physical and mental health, gastrointestinal symptoms, somatic symptoms, quality of life, anxiety and depression and faecal consistency (as measured by SF36, GSRS, PHQ12, BSFS, HADS and QoL questionnaires). Trial supplementation and equipment for collecting new samples is distributed.

At the end of the intervention (week 8), each patient has a visit with the medical team. A physical examination is done and symptoms (as measured by the diary, GSRS, PHQ12, IBS-SSS, BSFS, HADS and QoL scales etc.) are reassessed. Trial supplementation products are collected to check compliance. Faecal samples and blood samples are collected and analysed as before.

The primary end point is the mean change in the average number of headache days (days in which headache pain lasted ≥4 consecutive hours and had a peak severity of at least a moderate level or days in which acute migraine-specific medication [triptans or ergots] was used to treat a headache of any severity or duration) per month, comparing to the baseline 28-day screening period. The patients receiving the treatment product report a reduction in average number of headache days as compared to the placebo group. Further, where headache/migraine occurred, intensity is less in the treatment group. Secondary endpoints are the mean change in the average number of postdrome symptoms days and average symptom severity of postdrome symptoms, comparing to the baseline 28-day screening period. The patients receiving the treatment product report a reduction in average number of postdrome days and postdrome symptom severity as compared to the placebo group. The treatment group also indicate improved gastrointestinal symptoms as determined by GSRS score and an improvement in faecal consistency as compared to the placebo group. Patients having elevated anxiety scores as measured by HADS at baseline report a reduction in anxiety symptoms. Analysis of the blood indicates that the treatment patients have reduced levels of inflammatory markers, reduced gut permeability indicating an improved mucosal barrier, an increase in regulatory immune cells and a more balanced profile of metabolites from the kynurenine and serotonin pathways. The faecal analysis indicates that the treatment patients have reduced levels of bacterial overgrowth/dysbiosis and a higher level of bifidobacteria; especially members of the Bifidobacterium adolescentis phylogenetic group, Bifidobacterium longum and Bifidobacterium bifidum. Concentrations of short chain fatty acids are increased, and detrimental metabolites are decreased.

### Example 2

A total of 272 male and female participants who suffer from recurrent migraine are recruited from the general population to participate in the study. The participants complete a baseline screening survey where they indicate any medical conditions (including migraine), and various gastrointestinal and quality of life symptoms. For the symptoms, a 5-point Likert scale is used where a score of 1 means “No symptoms” and a score of 5 means “severe symptoms”. In the 272 participants, the following additional conditions are indicated:

| Condition | Number of participants |
|---|---|
| Irritable bowel syndrome | 86 |
| Diarrhoea | 141 |
| Constipation | 143 |
| Allergy | 137 |
| Food intolerance | 149 |
| Depression | 182 |

Each participant is provided with an amount of HMO sufficient for 3 weeks of a daily dose of about 4 g of HMO. The HMO is provided as either 2′-FL alone or a 4:1 mixture of 2′-FL and LNnT (by weight).

After 3 weeks of intake, each participant completes a second survey where they indicate various gastrointestinal and quality of life symptoms. The same 5-point Likert scale is used to assess the symptoms.

The process is repeated after 6 weeks, 9 weeks and 12 weeks.

Over the course to the 12 weeks, the headache/migraine participants indicate a reduction in headache/migraine occurrence and intensity. Further they indicate a reduction in postdrome occurrence and symptom severity and an improvement in gastrointestinal, quality of life and anxiety symptoms.

### Example 3

Paediatric patients of both sexes and of age between 5 and 15 are recruited. The patients all report at least 2 attacks of abdominal pain of at least 1 hour over the last month severe enough to interfere with daily activities. The attacks are accompanied by at least two of the following symptoms: anorexia, nausea, vomiting and pallor. There is complete resolution of symptoms between the attacks. Patients exhibiting symptoms of food intolerance, malabsorption or gastrointestinal and urinary tract disease diseases are excluded.

At an initial visit (screening), each patient and the parents/guardians are given both written and oral information about the study and the parents/guardians are asked to sign an informed consent form. Each patient is evaluated by a full review of clinical history including migraine history and undergoes a physical examination. Blood and urine samples are collected. Equipment for faecal sampling is distributed to each patient. The parents/guardians are instructed to keep the samples in the freezer until the next visit. The parents/guardians are provided with access to an electronic diary to record migraine occurrence and intensity. The NRS11 numerical pain scale is used to record pain levels. Patients also record associated migraine symptoms (e.g. nausea, photophobia and phonophobia), any medications used.

At a second visit (beginning of intervention) within 28 days of the first visit, eligibility criteria are checked, and eligible subjects are randomised to one of two arms. A total of 50 patients are included. Each arm has 25 patients, with one arm consuming the treatment product and one group the placebo product. The treatment product contains 5 grams of a combination of 2′-FL, DFL and LNnT while the placebo product contains 5 grams glucose. Both products are in powder form in a unit dosage container. The diary is reviewed, and an assessment is made of symptoms of physical and mental health, pain, gastrointestinal symptoms, somatic symptoms, quality of life, and faecal consistency (as measured by SF36, NRS11, GSRS, PHQ12, BSFS and QoL questionnaires). Trial supplementation is distributed. The faecal samples are collected and equipment for collecting new samples is distributed. Patients are instructed to maintain their current diet.

Blood samples and urine are collected for biomarker and biobanking. The serum from the blood samples is transferred to cryotubes and stored at −80° C. The following biomarkers are measured IFN-γ, TNF-α, IL-1β, IL-8, IL-6, IL-12, IL-10, MIP-1β, hs-CRP, lipopolysaccharide binding protein, fatty acid binding protein 2, tryptase, antiflagellin, zonulin, histamine, prostaglandin 2, and cortisol. To analysis the level of metabolites of the kynurenine and serotonin pathways following compounds were measured in serum; tryptophan, L-kynurenine, kynureninic acid and serotonin. Flow cytometry are performed on blood to determine the level of immune cells.

Urine samples are stored at −80° C. Bacterial metabolites such as SOFA are analysed in urine samples using NMR.

The faecal samples are stored at −80° C. until analysis. Microbiological analysis is performed on the faecal samples using the 16S rRNA gene sequence.

At a third visit after 6 weeks, the faecal samples are collected, blood and urine samples are collected, and an assessment is made of migraine occurrence and intensity, and of symptoms of physical and mental health, pain, gastrointestinal symptoms, somatic symptoms, quality of life, and faecal consistency (as measured by SF36, NRS11, GSRS, PHQ12, BSFS and QoL questionnaires). Trial supplementation and equipment for collecting new samples is distributed.

At the end of the intervention (week 12), each patient has a visit with the medical team. A physical examination is done and symptoms (as measured by the diary, NRS11, GSRS, PHQ12, IBS-SSS, BSFS and QoL scales etc.) are reassessed. Trial supplementation products are collected to check compliance. Faecal samples and blood samples are collected and analysed as before.

The primary end point is the mean change in the average number of abdominal pain days (days in which pain lasted hour and had a peak severity of at least a moderate level) per month, comparing to the baseline 28-day screening period. The patients receiving the treatment product report a reduction in average number of pain days as compared to the placebo group. Further, where pain occurred, intensity is less in the treatment group. Secondary endpoints are the mean change in the average number of postdrome symptoms days and average symptom severity of postdrome symptoms, comparing to the baseline 28-day screening period. The patients receiving the treatment product report a reduction in average number of postdrome days and postdrome symptom severity as compared to the placebo group. The treatment group also indicate improved gastrointestinal symptoms as determined by GSRS score and an improvement in faecal consistency as compared to the placebo group. Analysis of the blood indicates that the treatment patients have reduced levels of inflammatory markers, reduced gut permeability indicating an improved mucosal barrier, an increase in regulatory immune cells and a more balanced profile of metabolites from the kynurenine and serotonin pathways. The faecal analysis indicates that the treatment patients have reduced levels of bacterial overgrowth/dysbiosis and a higher level of bifidobacteria; especially members of the Bifidobacterium adolescentis phylogenetic group, Bifidobacterium longum and Bifidobacterium bifidum. Concentrations of short chain fatty acids are increased, and detrimental metabolites are decreased.

### Example 4

The HMOs 2′-FL and LNnT are introduced into a rotary blender in a 4:1 mass ratio. An amount of 0.25 mass % of magnesium stearate is introduced into the blender and the mixture blended for 10 minutes. The mixture is then agglomerated in a fluidised bed and filled into 5-gram stick packs and the packs sealed.

1-20. (canceled)

21. A method comprising
selecting a non-infant human experiencing two or more symptoms of episodic abdominal migraine;
selecting an effective amount of one or more human milk oligosaccharides (HMOs) chosen from the group consisting of 2′-fucosyllactose (2′-FL), difucosyllactose (DFL), 3-fucosyllactose (3-FL), lacto-N-fucopentaose I (LNFP-I), lacto-N-tetraose (LNT), lacto-N-neotetraose (LNnT), 3′-sialyllactose (3′-SL), 6′-sialyllactose (6′-SL), and combinations thereof, for reducing abdominal migraine symptoms in the non-infant human; and
reducing frequency of the two or more symptoms of episodic abdominal migraine by administering the effective amount of the chosen one or more HMOs.

22. The method of claim 21, wherein the non-infant human is a child of at least 5 years of age.

23. The method of claim 21, wherein the two or more symptoms of episodic abdominal migraine are selected from anorexia, nausea, vomiting, and pallor.

24. The method of claim 21, further comprising reducing severity of the one or more symptoms of episodic abdominal migraine by administering the effective amount of the chosen one or more HMOs.

25. The method of claim 21, further comprising reducing a level of gastrointestinal dysbiosis in the non-infant human and increasing the relative abundance of adult-type bifidobacteria species comprising one or more of B. adolescentis, B. longum, and B. bifidum, in the gastrointestinal microbiota of the non-infant human by administering the effective amount of the chosen one or more HMOs.

26. The method of claim 21, further comprising reducing frequency or severity of one or more symptoms of migraine headache in the non-infant human by administering the effective amount of the chosen one or more HMOs.

27. The method of claim 21, wherein the one or more symptoms of migraine headache reduced by administering the effective amount of the chosen one or more HMOs comprise predromal or postdromal symptoms selected from constipation, body aches, and food cravings.

28. The method of claim 21, wherein the one or more symptoms of migraine headache reduced by administering the effective amount of the chosen one or more HMOs comprise predromal or postdromal symptoms selected from fatigue, trouble concentrating, mental confusion, and depression.

29. The method of claim 21, wherein the one or more symptoms of migraine headache reduced by administering the effective amount of the chosen one or more HMOs comprise predromal or postdromal symptoms selected from skin sensitivity, scalp sensitivity, sensitivity to light, and sensitivity to sound.

30. The method of claim 21, wherein the chosen one or more HMOs comprise at least one fucosylated neutral HMO.

31. The method of claim 21, wherein the chosen one or more HMOs comprise at least one non-fucosylated neutral HMO.

32. The method of claim 21, wherein the chosen one or more HMOs comprise at least one sialylated HMO.

33. The method of claim 21, wherein the chosen one or more HMOs comprise a mixture of HMOs selected from:
a mixture of 2′-FL, DFL, LNT and LNnT;
a mixture of 3′-SL and 6′-SL; and
a mixture of 2′-FL, DFL, LNT, LNnT, 3′-SL and 6′-SL.

34. The method of claim 21, wherein the effective amount of the chosen one or more HMOs is a daily dose of from about 3 g to about 15 g during an initial treatment phase.

35. The method of claim 34, wherein a duration of the initial treatment phase is at least two weeks.

36. The method of claim 35, wherein the effective amount of the chosen one or more HMOs is a daily dose of from about 2 g to about 5 g during a maintenance treatment phase subsequent to the initial treatment phase.

37. A method comprising
selecting a non-infant human experiencing one or more prodromal or postdromal symptoms of migraine headache;
selecting an effective amount of one or more human milk oligosaccharides (HMOs) chosen from the group consisting of 2′-fucosyllactose (2′-FL),
difucosyllactose (DFL), 3-fucosyllactose (3-FL), lacto-N-fucopentaose I (LNFP-I), lacto-N-tetraose (LNT), lacto-N-neotetraose (LNnT), 3′-sialyllactose (3′-SL), 6′-sialyllactose (6′-SL), and combinations thereof, for reducing abdominal migraine symptoms in the non-infant human; and
reducing frequency or severity or both of the one or more prodromal or postdromal symptoms of migraine headache by administering the effective amount of the chosen one or more HMOs.

38. The method of claim 37, wherein the chosen one or more HMOs comprise a mixture of at least one fucosylated neutral HMO and at least one nonfucosylated neutral HMO.

39. The method of claim 37, wherein the one or more prodromal or postdromal symptoms of migraine headache are selected from skin sensitivity, scalp sensitivity, sensitivity to light, and sensitivity to sound.

40. The method of claim 37, further comprising reducing a level of gastrointestinal dysbiosis in the non-infant human and increasing the relative abundance of adult-type bifidobacteria species comprising one or more of B. adolescentis, B. longum, and B. bifidum, in the gastrointestinal microbiota of the non-infant human by administering the effective amount of the selected one or more HMOs.
